(19) United States
(12) Patent Application Publication
(10) Pub. No.: US 2003/0180827 A1
(43) Pub. Date: Sep. 25, 2003

(54) Highly sensitive and continuous protein tyrosine phosphatase test using 6,8-difluoro-4-methylumbelliferyl phosphate

(75) Inventors: Stefan Welte, Neuberg (DE); Norbert Tennagels, Frankfurt (DE); Stefan Petry, Frankfurt (DE)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma Deutschland Gmbh.

(21) Appl. No.: 10/334,081

(22) Filed: Dec. 30, 2002

Related U.S. Application Data
(60) Provisional application No. 60/357,885, filed on Feb. 19, 2002.

(30) Foreign Application Priority Data
Jan. 4, 2002 (DE) 10200173.1
Aug. 8, 2002 (DE) 10236329.3

(51) Int. Cl. C12Q 1/42

(52) U.S. Cl. 435/21

(57) ABSTRACT

The invention describes a method for measuring a protein tyrosine phosphatase in biological material using 6,8-difluoro-4-methylumbelliferyl phosphate (“DiFMUP”).

### RELATED APPLICATIONS

This application claims benefit of U.S. Provisional Application No. 60/357,885, filed Feb. 19, 2002, and German Patent Applications 10200173.1, filed Jan. 4, 2002, and 10236329.3, filed Aug. 8, 2002 and are herein incorporated by reference.

### FIELD OF THE INVENTION

The invention relates to an improved method for measuring the activity of protein tyrosine phosphatases (“PTPs”) using 6,8-difluoro-4-methylumbelliferyl phosphate (“DiFMUP”).

### BACKGROUND OF THE INVENTION

The prior art discloses methods for measuring the activity of a protein tyrosine phosphatase using the substrate p-nitrophenyl phosphate (“p-NPP”). These methods are to be found in standard biochemical manuals such as Current Protocols in Protein Science: John E. Coligan (ed.), Ben M. Dunn (ed.), Hidde L. Ploegh (ed.), David W. Speicher (ed.), Paul T. Wingfield (ed.); SBN: 0-471-11184-8; loose-leaf pages, continuously updated; published by John Wiley & Sons. In these methods, the action of a phosphatase forms p-nitrophenol from the p-NPP. The p-nitrophenol thus produced can be measured photometrically on the basis of its intense yellow color in the alkaline range.

However, this test method has some unfavorable features. The test is not suitable for directly determining the activity of the enzyme since it is carried out in accordance with the time-stop principle. In accordance with this principle, the enzyme reaction is interrupted by adding sodium hydroxide solution after a given period of time has elapsed. The increase in the pH leads to the color of the resulting p-nitrophenol changing to yellow, the absorption of which is determined photometrically and is a measure of the quantity of p-nitrophenol which is present. This test principle is rather elaborate for enzyme-kinetic investigations, for example for determining the type of inhibition. Since a large number of experimental points are required, a corresponding number of individual assays, which are coordinated with each other, have to be set up.

In addition, the p-NPP substrate is sensitive to light, temperature, and pH. In the physiological pH range, it has a tendency to decompose slowly. When p-NPP is used as the substrate, some of the tyrosine phosphatases to be investigated have an activity maximum which is in the acid range. For example, the tyrosine phosphatase PTP1B shows a greater turnover at a pH of 5.6. On the other hand, at the physiological pH of 7.0, PTP1B is only operating at 30% of the maximum turnover value when using p-NPP as its substrate. This makes it necessary to use relatively high quantities of the enzyme, resulting in a corresponding increase in costs.

Another negative factor of this test method is that other components which are present in the test, such as buffers, salts or other substances (test substances), from time to time absorb in the yellow range. This requires appropriate background controls.

The malachite green phosphopeptide test has been described as another method for measuring the activity of protein tyrosine phosphatases. Martin et al. (1985) Journal of Biological Chemistry, 260, pp. 14932 and Harder et al. (1994) Biochemical Journal, 298, pp. 395. In this method, the inorganic phosphate which the phosphatase has released from its substrate peptide is detected photometrically using the malachite green reagent. Apart from the temperamental nature of the photometric determination, resulting, for example, from impurities or pH sensitivity, and the laboriousness of the time-stop method, another disadvantage is that the specific substrate peptide has to be prepared in high concentration for each phosphatase, something which generally makes the method rather expensive.

Another substrate that has been described in the prior art is 3,6-Fluorescein diphosphate (Journal of Biomolecular Screening 4, 327-334, 1999). While this substrate, in contrast to the two substrates mentioned above, enables the enzyme activity to be measured directly, the spectral properties of this substrate are still not particularly good for carrying out measurements in the physiological pH range.

The object of the present invention is therefore to make available another method for testing for protein tyrosine phosphatases, which method is more reliable and more economical in use.

### SUMMARY OF THE INVENTION

The invention relates to a method for measuring the enzymic activity of a protein tyrosine phosphatase (“PTP”) in biological material, which comprises a providing a PTP, providing 6,8-difluoro-4-methylumbelliferyl phosphate (“DiFMUP”), contacting the PTP to the DiFMUP in an aqueous solution, and fluorometrically measuring the 6,8-difluoro-4-methylumbelliferyl which is then formed.

In one aspect, the protein tyrosine phosphatase is selected from the group consisting of: LAR, CD 45, PTP alpha, PTP 1B, TCPTP, YOP, CDC 25, PTEN and SHP1,2. The PTP can be present in different stages of purity. The PTP can be provided in a preparation obtained from whole cells, disintegrated cells, samples enriched with cell components and/or organelles, or purified proteins.

In one aspect, when being brought into contact with the PTP, the concentration of the DiFMUP in the aqueous solution is from 10 to 250 μM. In a further aspect, the concentration of the DiFMUP is from 50 to 100 μM.

In one aspect, the pH of the aqueous solution is between 5.0 and 8.0. In a further aspect, the pH of the aqueous solution is between 6.0 and 7.5. In yet a further aspect, the pH is 7.0.

The invention also relates to a method for identifying a compound which modifies the activity of a PTP, which comprises measuring PTP activity using DiFMUP as a substrate both in the presence a test compound and in a control assay, and identifying the test compound as an effector if the two measurements yield significantly different results. A control assay is characterized, in particular, by the fact that, when the biological material or the preparation obtained from biological material is brought into contact with DiFMUP, either no chemical compound is involved or the effect of the chemical compound in relation to a protein tyrosine phosphatase of the chosen type is already known. Such chemical compounds, which are used in the control assay and whose effect on a protein tyrosine phosphatase is already known, can, in particular, be vanadate, vanadium organic compounds, pervanadate, okadaic acid, NaF, dephostasin, modified peptides or other compounds.

In one aspect, the method for identifying a compound which modifies the activity of a protein tyrosine phosphatase, the modification should comprise a stimulation, inhibition or stabilization of the activity of a protein tyrosine phosphatase.

In one aspect, the method for identifying a compound which modifies the activity of a protein tyrosine phosphatase, this protein tyrosine phosphatase is selected from the group consisting of: leucocyte antigen-related protein tyrosine phosphatase (“LAR”), leucocyte phosphatase CD 45 (“CD 45”), yersinia protein tyrosine phosphatase (“YOP”), protein tyrosine phosphatase alpha (“PTP alpha”), protein tyrosine phosphatase 1B (“PTP 1B”), T cell-protein tyrosine phosphatase (“TC-PTP”), cell-division-control phosphatase 25 (“CDC25”), phosphatase (dual specific) within chromosome 10 (“PTEN”), and src-homology phosphatase 1,2 (“SHP 1,2”).

The invention also relates to a compound which modifies the activity of a protein tyrosine phosphatase and which has been identified by the above-described methods. In one aspect, the compound has a mass of between 0.1 and 50 kDa. In a further aspect, the compound has a mass of between 0.1 and 5 kDa and in yet a further aspect the compound has a mass of between 0.1 and 3 kDa. The compound can be a protein, an amino acid, a polynucleotide, a nucleotide, a natural product or an aromatic hydrocarbon compound.

The invention also relates to a pharmaceutical which comprises at least one compound identified by a method as described above, and a pharmaceutically acceptable carrier. In one aspect, the pharmaceutically acceptable carrier comprises auxiliary substances for formulating a pharmaceutical. In another aspect, the pharmaceutically acceptable carrier comprises polymeric additives.

The invention also relates to the use of a compound, which has been identified by a method for identifying a compound which modifies the activity of a protein tyrosine phosphatase, for producing a pharmaceutical for treating diabetes.

### BRIEF DESCRIPTION OF THE FIGURES

FIG. 1 is a graph depicting cleavage of DiFMUP at various concentrations of the PTP1B enzyme.

FIG. 2 is a graph depicting cleavage of DiFMUP at various concentrations of the PTP1B enzyme.

FIG. 3 comprises six graphs depicting cleavage of DiFMUP at various concentrations of PTPalpha, LAR, TCPTP, SHP 2, CD45 and YOP.

FIG. 4 is a graph depicting the inhibitory effect exerted by a phosphatase inhibitor of PTP1B.

FIG. 5 comprises two graphs contrasting reversible and non-reversible inhibition types of a phosphatase inhibitor

FIG. 6 comprises two graphs contrasting reversible and non-reversible inhibition types of a phosphatase inhibitor

### DETAILED DESCRIPTION OF THE INVENTION

6,8-Difluoro-4-methylumbelliferyl phosphatase is commercially available. For example, the company Molecular Probes Europe BV (2333 Leiden, The Netherlands) markets this chemical. Its preparation is disclosed in U.S. Pat. No. 5,830,912.

Protein tyrosine phosphatases (“PTPs”) can be prepared from biological material. Biological material is any material which contains genetic information and which is able to reproduce itself or to be reproduced in a biological system. Examples of biological material are cells from human or animal tissues or organs, such as, in particular, brain, fatty tissue, lung, heart, liver, kidney, spleen, muscle and others. Examples of biological material include bacteria such as Escherichia coli and fungi such as Saccharomyces cerevisiae. Biological material also comprises cells from cell cultures. In the case of cells from animal or human tissues, biological material can be obtained by biopsy, surgical removal or removal using syringes or catheters, or comparable techniques. The cells which have been removed in this way can be deep-frozen, worked up or taken into culture. Bacteria and yeast cells are propagated using customary microbiological techniques and worked up. The skilled person will find appropriate instructions for this purpose in “Current Protocols in Molecular Biology; ed.: F. M. Ansabel et al., loose-leaf publication, continuously updated, 2001 edition, published by John Wiley & Sons”.

Biological material can also comprise the cells from a culture of animal cells. Examples of such cells are mouse cells, rat cells or hamster cells. The cell culture can be primary cell types or established cell lines. Examples of established cell lines are mouse 3T3 cells, CHO cells, and Hela cells. The maintenance, growth, and propagation of cell lines is described in standard textbooks, for example in “Basic Cell Culture; ed.: J. M. Daris IRL Press, Oxford (1996)”.

A PTP preparation obtained from a biological material is prepared, for example, by the disruption of the biological material and by subsequent purification steps. Methods for disrupting the biological material can be, in particular, repeated freezing and thawing, sonication, the use of a French press or the addition of detergents and enzymes, or the like. Subsequent purification steps comprise, for example, differential centrifugation, precipitation with ammonium sulfate or organic solvents, the use of chromatographic techniques, etc. Examples of chromatographic techniques are polyacrylamide gel electrophoresis, high pressure liquid chromatography, ion exchange chromatography, affinity chromatography, gas chromatography, mass spectrometry, etc. Textbooks, such as, in particular, “Current Protocols in Protein Science, ed.: J. E. Coligan et al., loose-leaf publication, continuously updated, 2001 edition, published by John Wiley & Sons” are available to the skilled person for this purpose and, in particular, also for detailed instructions with regard to purifying proteins.

The PTP preparation can be brought into contact with DiFMUP in an aqueous medium in customary laboratory vessels such as Eppendorf tubes, centrifuge tubes or glass flasks. In one embodiment, the aqueous medium contains, buffering substances, nutrient medium constituents, singly charged or doubly charged ions such as Na+, K+, Ca2+, Cl−, SO42− and PO32−, or other ions, and, in addition, proteins, glycerol or other substances. For contacting the PTP to the DiFMUP, specific constant conditions, such as the temperature, pH, ionic conditions, protein concentration, and volume, can be advantageous. This is achieved by, for example, contacting the PTP to the DiFMUP in incubation devices which are kept at a constant temperature, in the presence of a buffer or using quantities of the ions or proteins which have previously been weighed out accurately. The aqueous solvent can, in particular, also contain a particular proportion of an organic solvent, such as dimethyl sulfoxide, methanol or ethanol. However, the content of such a solvent is preferably not more than 10% by volume of the mixture.

The PTP protein family currently comprises about 100 different members. These members can be roughly subdivided into receptor-coupled proteins and cytoplasmic proteins. The phosphatases possess in common the amino acid motif (H/V)CX5R(S/T) in the catalytic domain. The receptor-coupled phosphatases usually comprise an extracellular domain, a single transmembrane region and one or two cytoplasmic PTP domains. The LAR (leukocyte common antigen-related) protein and the PTPα proteins are considered to belong to the receptor-coupled PTPs. The intracellular PTPs normally contain a catalytic domain and various extensions of the C-terminal or N-terminal region, for example as a result of “SH domains”. These extensions are ascribed functions in targeting or regulation. The enzyme PTP1B is assigned to the cytoplasmic PTPs. In addition to the pure tyrosine phosphatases, the PTP family also includes the group of dual phosphatases. In addition to phosphotyrosine, these enzymes also use phosphoserine or phosphothreonine as the substrate. This group includes, for example, the phosphatases VHR and cdc25.

The phosphatases LAR, PTPα, SHP-2 and PTP1B are ascribed important functions in the insulin-mediated signal pathway. These PTPs associate with the insulin receptor and catalyze dephosphorylation. These PTPs may possibly play a role, individually or in combination, in the pathogenesis of insulin resistance (Biochemistry 38, 3793-3803, 1999; p. 3799).

PTP1B is a negative regulator of the insulin-stimulated signal transduction pathway, i.e. the protein once again switches off the signal which was induced by insulin. Presumably, the signal pathway is interrupted by the insulin receptor being directly dephosphorylated. PTP1B is also overexpressed in a large percentage of patients suffering from breast cancer. In addition, the enzyme interacts with the “epidermal growth factor”. The enzyme has been demonstrated to possess two aryl phosphate binding pockets. One is located directly in the active center while another is located outside of this at a site which is adjacent to the catalytic center. Biochemistry 38, 3793-3803, 1999.

The transmission and termination of many intracellular signals is controlled by the tyrosine-phosphorylation of the factors involved. A precisely balanced activity of complementary protein tyrosine kinases (PTKs) and phosphatases, in particular PTPs, establishes the phosphorylation state. As enzymes, PTPs are responsible for selectively dephosphorylating phospho-tyrosine residues. PTPs function, in interplay with the protein tyrosine kinases, in a great variety of different biological processes, in mediating signals due, for example, to growth factors or hormones. These signal transduction mechanisms play an important role in regulating cell metabolism, growth, differentiation or mobility. The faulty regulation of signal pathways is thought to be one of the causes of a number of pathological processes. These processes include, for example, cancer, some immunological and neurological diseases, and also type II diabetes and obesity.

A chemical compound is provided, for example, by means of chemical synthesis. The skilled person is familiar with the standard methods of synthesis. The chemical compound can be part of a collection of chemical compounds, as are formed by storing and cataloging the chemical compounds from synthesis programs which have been concluded (what are termed chemical libraries). In other cases, the compound can have been formed by a microorganism, in particular a bacterium, or else by a fungus or a plant (natural product).

Suitable pharmaceutical compounds for oral administration can be present in separate units, such as capsules, cachets, sucking tablets or tablets, as powders or granules, as a solution or a suspension in an aqueous or non-aqueous liquid, or as an oil-in-water or water-in-oil emulsion. These compositions can be prepared in accordance with any suitable pharmaceutical method which comprises a step in which the active compound and the excipient (which can be composed of one or more additional constituents) are brought into contact. In general, the compositions are prepared by uniformly and homogeneously mixing the active compound with a liquid and/or finely divided solid excipient, after which the product is formed, if required. Thus, a tablet can be prepared, for example, by pressing or forming a powder or granulate of a compound, where appropriate together with one or more additional constituents. Pressed tablets can be prepared by tableting the compound in freely flowing form, such as a powder or granulate, where appropriate mixed with a binder, a glidant, an inert diluent and/or a (several) surface-active/dispersing agent(s), in a suitable machine. Formed tablets can be prepared by forming the pulverulent compound, which has been moistened with an inert liquid diluent, in a suitable machine.

Pharmaceutical compositions which are suitable for peroral (sublingual) administration comprise sucking tablets which contain a compound together with a flavoring substance, customarily sucrose and gum arabic or tragacanth, and lozenges, which comprise a compound in an inert base such as gelatin and glycerol or sucrose and gum arabic.

Suitable pharmaceutical compositions for parenteral administration preferably comprise sterile aqueous preparations of a compound, which preparations are preferably isotonic with the blood of the intended recipient. These preparations are preferably administered intravenously, even if the administration can also take place subcutaneously, intramuscularly or intradermally as an injection.

These preparations can preferably be produced by mixing the compound with water and making the resulting solution sterile and isotonic with blood. Injectable compositions according to the invention generally comprise from 0.1 to 5% by weight of an active compound.

Suitable pharmaceutical compositions for rectal administration are preferably present in the form of single-dose suppositories. These can be produced by mixing a compound with one or more conventional solid excipients, for example cocoa butter, and forming the resulting mixture.

Suitable pharmaceutical compositions for topical use on the skin are preferably present in the form of an ointment, cream, lotion, paste, spray, aerosol or oil. Excipients which can be used are Vaseline, lanolin, polyethylene glycols, alcohols and combinations of two or more of these substances. The active compound is generally present at a concentration of from 0.1 to 15% by weight of the composition, for example of from 0.5 to 2%.

A transdermal administration is also possible. Suitable pharmaceutical compositions for transdermal uses can be present as individual plasters which are suitable for close, long-term contact with the epidermis of the patient. Such plasters suitably comprise the active compound in an aqueous solution which is buffered, where appropriate, dissolved and/or dispersed in an adhesive or dispersed in a polymer. A suitable active compound concentration is from approx. 1% to 35%, preferably from approx. 3% to 15%.

Type 2 diabetes (NIDDM—non-insulin-dependent diabetes mellitus) is characterized by high glucose values (hyperglycemia) in the fasting state (>126 mg/dl), insulin resistance in peripheral tissues such as muscle or fat, an increase in gluconeogenesis in the liver and inadequate secretion of insulin by the pancreatic β cells. The actual cause of this disease is still not known. Type 2 diabetes very frequently occurs together with other clinical pictures, such as obesity, hypertriglyceridemia (elevated blood fat values) and high blood pressure.

Insulin resistance is suspected to be a key to understanding the clinical picture. Insulin resistance is expressed in a decreased ability of the peripheral organs to react to a defined concentration of insulin. This is reflected at the cellular level, i.e. in an increase in the quantity of insulin which is required for inducing an effect due to insulin. In muscle, fat and liver cells, insulin has a variety of effects on glucose metabolism and fat metabolism, such as increasing the uptake of glucose from the blood, increasing the rate at which glucose is metabolized or inhibiting fatty acid cleavage. A variety of factors are thought to have a fundamental connection with the development of insulin resistance at the cellular level. The insulin receptor, the factors of the signal cascade, and the components of the glucose transport system, play an important role in this context.

Insulin brings about its biological functions by, in the first step, binding to the insulin receptor. After this receptor has bound the insulin, its β subunit is subjected to autophosphorylation by the insulin receptor kinase. In muscle cells, the signal is cellularly transmitted by way of the IRS (insulin receptor substrate) and PI3K (phosphoinositol-3-kinase) and leads to glucose uptake being stimulated. Insulin brings about a large number of other effects which proceed by way of mechanisms which are only partially understood. In the intracellular transmission of the signal, specific kinases and phosphatases act together in a coordinated manner. Dissociation of the insulin from the receptor is not sufficient to switch off the signal induced by the insulin. The tyrosine kinase activity of the insulin receptor persists as long as the regulatory domain remains phosphorylated. Cellular PTPs are responsible for switching off the signal. Pharmacologically active compounds which have an inhibitory effect on negative regulators of the insulin signal pathway have the potential to delay dephosphorylation of the insulin receptor. This provides the possibility of being able to use the substances for decreasing the resistance to insulin.

### EXAMPLES

1. Cleavage of DiFMUP in Dependence on the Concentration of the PTP1B Enzyme

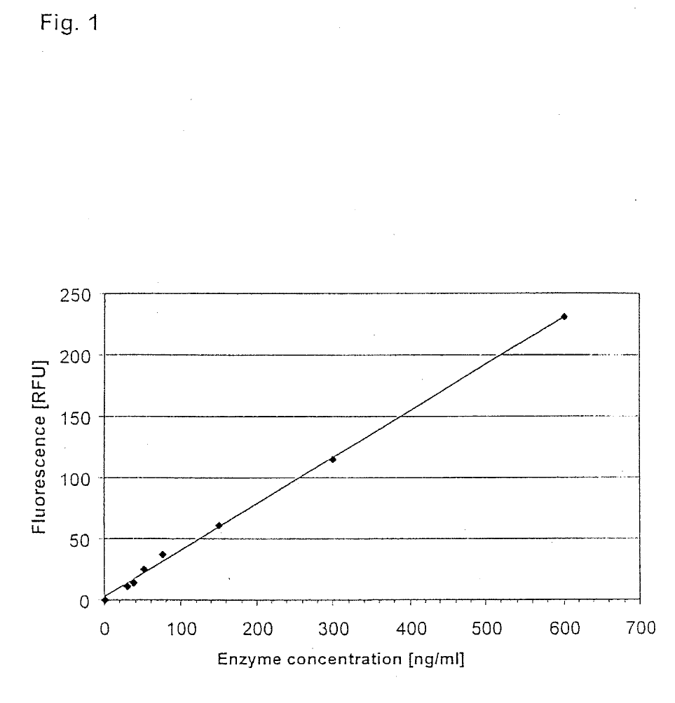

The reaction takes place in a black microtiter plate at a temperature of 37° C. 135 μl of reaction buffer are provided per enzyme concentration to be analyzed, with this reaction buffer containing the following components: protein tyrosine phosphatase PTP1B at the desired final concentration (FIG. 1: 30-600 ng/ ml); 50 mM Hepes, pH 6.9; 150 mM NaCl; 1 mM EDTA; 2 mM DTT. The phosphatase reaction is started by adding 15 μl of 1 mM DiFMUP solution and the increase in fluorescence (measured in RFU) is measured, continuously for 15 minutes, in a fluorescence microtiter plate photometer at an excitation wavelength of 358 nm and an emission wavelength of 455 nm. The measure of the enzyme activity is the increase in fluorescence in dependence on the final concentration of PTP1B, which can be depicted graphically (FIG. 1).

2. Concentration Dependence of the Cleavage of DiFMUP by PTP1B

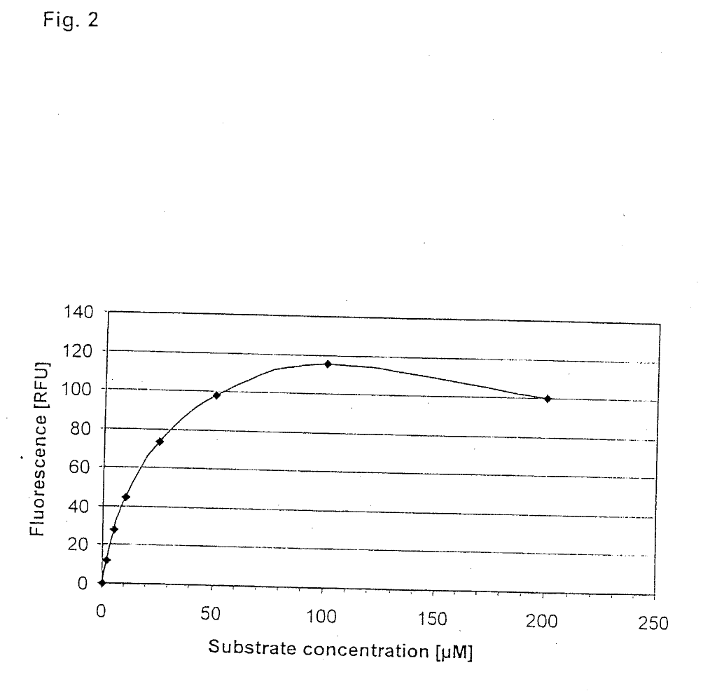

The reaction takes place in a black microtiter plate at a temperature of 37° C. 135 μl of reaction buffer are provided in each case, with this buffer containing the following components: 100 ng of protein tyrosine phosphatase PTP1b/ml; 50 mM Hepes, pH 6.9; 150 mM NaCl; 1 mM EDTA; 2 mM DTT. The phosphatase reaction is started by adding 15 μl of DiFMUP solution, which contains the substrate at 10 times the final concentration which is desired in the test mixture (FIG. 2: 0-200 μM), and the fluorescence is measured, at time intervals of 30 seconds for a period of 15 minutes, in a fluorescence microtiter plate photometer at 358/455 nm. The measure of the enzyme activity is the increase in fluorescence (measured in RFU) in dependence on the DiFMUP concentration, which can be depicted graphically (FIG. 2). This graph can subsequently be used to determine the kinetic constants of the enzyme reaction by means of a Lineweaver-Burk analysis. Thus, PTP1B is found to have a Km value of 19 μM and a Vmax of 388000 RFU sec-1 mg-1. This analysis can also be carried out in an analogous manner for other tyrosine phosphatases. The kinetic constants are given in Table 1.

3. Cleavage of DiFMUP in Dependence on the Concentration of the Phosphotyrosine Phosphatase Enzymes PTPalpha, LAR, T cell-PTP, SHP-2, CD45 and YOP.

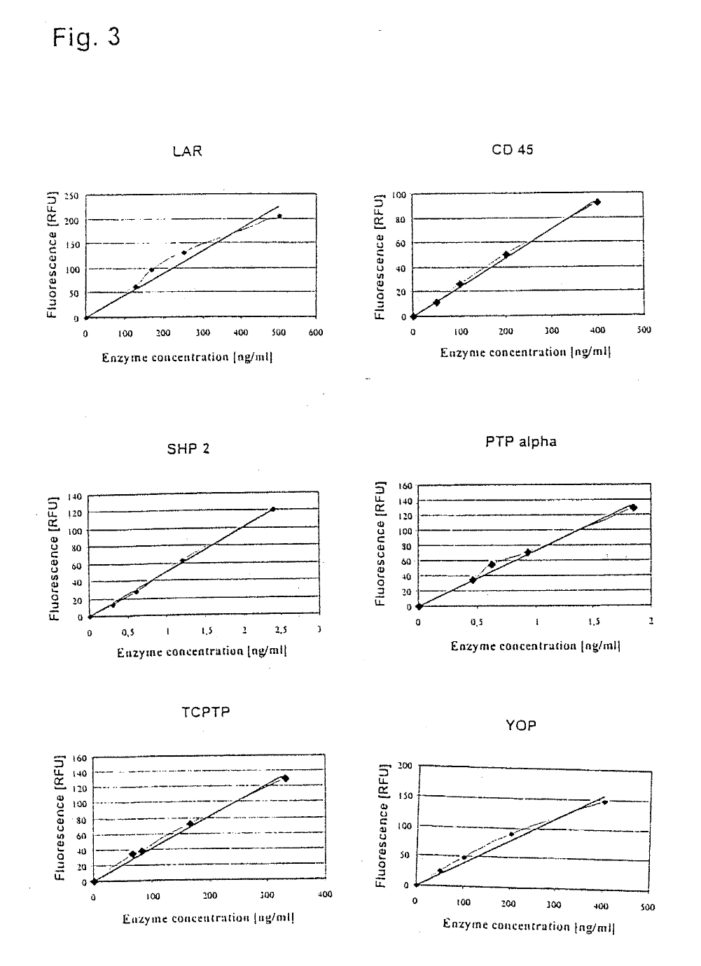

The reaction takes place in a black microtiter plate at a temperature of 37° C. 135 μl of reaction buffer are provided per enzyme and per enzyme concentration to be analyzed, with this buffer containing the following components: protein tyrosine phosphatase at the desired final concentration (FIG. 3: PTPalpha: 0.5-1.85 μg/ml, LAR:125-500 ng/ml; Tcell-PTP:66-330 ng/ml; CD 45:50-400 ng/ml; YOP:50-400 ng/ml; SHP-2:0.3-2.4 μg/ml); 50 mM Hepes, pH 6.9; 150 mM NaCl; 1 mM EDTA; 2 mM DTT.

The phosphatase reaction is started by adding 15 μl of 1 mM DiFMUP solution and the fluorescence is measured, at time intervals of 30 seconds and over a period of 15 minutes, in a fluorescence microtiter plate photometer at 358/455 nm. The measure of the enzyme activity is the increase in fluorescence (measured in RFU) in dependence on the final concentrations of the protein tyrosine phosphatases, which can be depicted graphically (FIG. 3).

4. Determining the Inhibitory Effect of a Phosphatase Inhibitor of PTP1B.

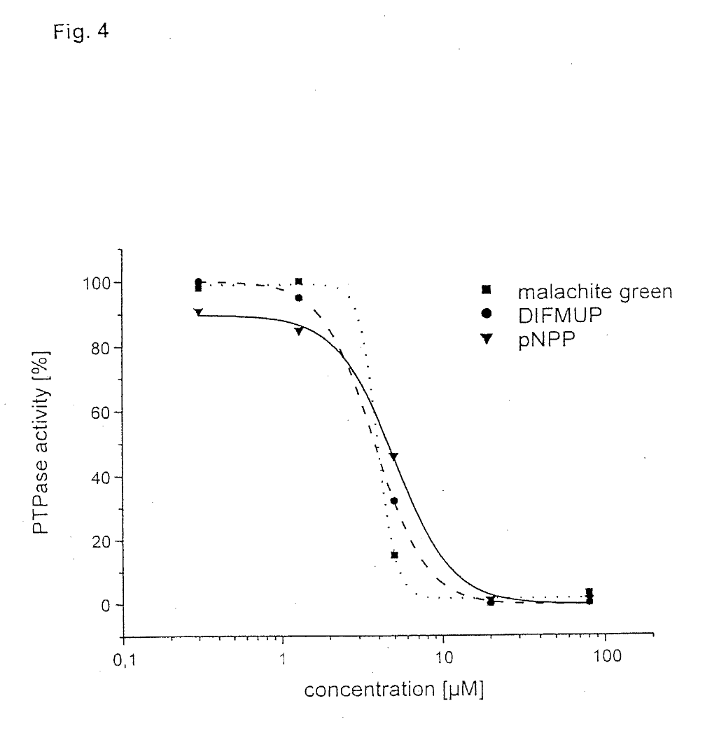

The test for determining the inhibitory effect of the active compound 2,2-dioxo-2,3dihydro-2,6-benzo[1,2,3]oxathiazol-5-yl)-(9-ethyl-9H-carbazol-3-ylmethyl)amine using the DiFMUP test takes place in a black microtiter plate at a temperature of 37° C. 120 μl volumes of reaction buffer are provided, with the buffer containing the following components: 100 ng of protein tyrosine phosphatase PTP1B/ml; 50 mM Hepes, pH 6.9; 150 mM NaCl; 1 mM EDTA; 2 mM DTT. To this are added 15 μl of the inhibitor solution to be tested, at a variety of concentrations. The phosphatase reaction is started by adding 15 μl of 1 mM DiFMUP solution and the fluorescence (measured in RFU) is measured, at time intervals of 30 seconds and over a period of 15 minutes, in a fluorescence microtiter plate photometer at 358/455 nm. The measure of the enzyme activity is the increase in fluorescence, which can be depicted graphically (FIG. 1). The reduction in enzymic activity which is obtained depends on the concentration of inhibitor employed. The inhibitor concentration at which the active compound 2,2-dioxo-2,3-dihydro-2,6-benzo[1,2,3]oxathiazol-5-yl)-(9-ethyl-9H-carbazol-3-ylmethyl)amine reduces the activity of the PTP1B by half (IC-50) can be determined to be 3.8 μM. The IC-50 values using the pNPP test method and the malachite green phosphopeptide test were determined for comparison. In this connection, the pNPP test method gives an IC50 value of 5.1 μM and the malachite green phosphopeptide test method gives an IC50 value of 3.9 μM. The corresponding inhibition curves are included in FIG. 4.

5. Characterizing the Type of Inhibition Exerted by a Phosphatase Inhibitor on PTP1b.

The reaction takes place in a black microtiter plate at a temperature of 37° C. 120 μl volumes of reaction buffer are provided, with the buffer containing the following components: 100 ng of protein tyrosine phosphatase PTP1b/ml; 50 mM Hepes, pH 6.9; 150 mM NaCl; 1 mM EDTA; 2 mM DTT and an inhibitor concentration which depends on the previously determined IC50. The phosphatase reaction is started by adding 15 μl of DiFMUP solution, which contains the substrate at 10 times the desired final concentration in the final volume (FIG. 2: 0-200 μm), and the fluorescence is measured, at time intervals of 30 seconds for a period of 15 minutes, in a fluorescence microtiter plate photometer at 358-455 nm until the reaction goes into saturation. Subsequently, a 10-fold excess of the previously employed final concentration of substrate is added and the reaction continues to be monitored, at time intervals of 30 seconds for a period of 15 minutes, in a fluorescence microtiter plate photometer at 358-455 nm. The reaction cannot be restarted in the presence of irreversible inhibitors while it is possible to do this when the inhibition is of the reversible type (FIG. 5).

6. Characterizing, by Means of Time-Dependent Incubation, the Type of Inhibition Exerted by a Phosphatase Inhibitor on PTP1b.

The reaction takes place in a black microtiter plate at a temperature of 37° C. 120 μl volumes of reaction buffer are provided, with the buffer containing the following components: 100 ng of protein tyrosine phosphatase PTP1b/ml; 50 mM Hepes, pH 6.9; 150 mM NaCl; 1 mM EDTA; 2 mM DTT and an inhibitor concentration which depends on the previously determined IC50. The mixture is incubated and, at defined points in time, the phosphatase reaction is started by adding 15 μl of DiFMUP solution, which contains the substrate at 10 times the desired final concentration in the final volume (FIG. 2: 0-200 μM), and the fluorescence is measured, at time intervals of 30 seconds for a period of 15 minutes, in a fluorescence microtiter plate photometer at 358-455 nm.

In the presence of irreversible inhibitors, the decrease in enzyme activity depends on the preincubation time, whereas this phenomenon cannot be observed in the case of inhibitors of the reversible inhibition type (FIG. 6).

What is claimed is:

1. A method for measuring the enzymic activity of a protein tyrosine phosphatase, said method comprising the steps of:
(a) providing a protein tyrosine phosphatase;
(b) providing 6,8-difluoro-4-methylumbelliferyl phosphate (“DiFMUP”);
(c) contacting said protein tyrosine phosphatase with said DiFMUP in an aqueous solution; and
(d) fluorometrically measuring 6,8-difluoro-4-methylumbelliferyl.

2. The method of claim 1, wherein said protein tyrosine phosphatase is selected from the group consisting of: leucocyte antigen-related protein tyrosine phosphatase (“LAR”), leucocyte phosphatase CD 45 (“CD 45”), yersinia protein tyrosine phosphatase (“YOP”), protein tyrosine phosphatase alpha (“PTP alpha”), protein tyrosine phosphatase 1B (“PTP1B”), T cell-protein tyrosine phosphatase (“TC-PTP”), cell-division-control phosphatase 25 (“CDC-25”), phosphatase (dual specific) within chromosome 10 (“PTEN”), and src-homology phosphatase 1,2 (“SHP 1,2”).

3. The method of claim 1, wherein said DiFMUP is at a concentration between 10 and 250 μM in said aqueous solution.

4. The method of claim 3, wherein said DiFMUP is at a concentration between 50 and 100 μM in said aqueous solution.

5. The method of claim 1, wherein the pH of said aqueous solution is between 5.0 and 8.0.

6. The method of claim 5, wherein the pH of said aqueous solution is between 6.0 and 7.5.

7. The method of claim 6, wherein the pH of said aqueous solution is 7.0.

8. A method for identifying a test substance as an effector of a protein tyrosine phosphatase, said method comprising the steps of:
(a) measuring protein tyrosine phosphatase activity in the presence of said test substance according to the method of claim 1;
(b) measuring protein tyrosine phosphatase activity in a control assay according to the method of claim 1;
(c) identifying said test substance as an effector if said measuring step (a) yields a significantly different result than said measuring step (b).

9. The method of claim 8, wherein said control assay comprises measuring protein tyrosine phosphatase activity in the absence of said test substance according to the method of claim 1.

10. The method of claim 8, wherein said test substance stimulates, inhibits, or stabilizes the activity of said protein tyrosine phosphatase.

11. The method of claim 8, wherein said protein tyrosine phosphatase is selected from the group consisting of: leucocyte antigen-related protein tyrosine phosphatase (“LAR”), leucocyte phosphatase CD 45 (“CD 45”), yersinia protein tyrosine phosphatase (“YOP”), protein tyrosine phosphatase alpha (“PTP alpha”), protein tyrosine phosphatase 1B (“PTP 1B”), T cell-protein tyrosine phosphatase (“TC-PTP”), cell-division-control phosphatase 25 (“CDC-25”), phosphatase (dual specific) within chromosome 10 (“PTEN”), and src-homology phosphatase 1,2 (“SHP 1,2”).

12. An effector compound identified by the method of claim 8.

13. The effector compound of claim 12, wherein the mass of said compound is between 0.1 and 50 kDa.

14. The effector compound of claim 13, wherein the mass of said compound is between 0.1 and 5 kDa.

15. The effector compound of claim 14, wherein the mass of said compound is between 0.1 and 3 kDa.

16. The effector compound of claim 12, wherein said compound is selected from the group consisting of: a protein, an amino acid, a polysaccharide, a sugar, a polynucleotide, a nucleotide, a natural product, and an aromatic hydrocarbon compound.

17. A pharmaceutical comprising at least one compound as claimed in claim 12, and a pharmaceutically suitable carrier.

18. The pharmaceutical of claim 17 wherein said pharmaceutically suitable carrier comprises polymeric additives.

19. A method for producing a pharmaceutical for treating diabetes comprising the step of providing a compound as claimed in claim 12.
